(19) United States
(12) Patent Application Publication
(10) Pub. No.: US 2004/0002649 A1
(43) Pub. Date: Jan. 1, 2004

(54) Instrument-holding projection imaging vector guide and method of use

(75) Inventor: Donald W. Chakeres, Columbus, OH (US)

Correspondence Address: STANDLEY LAW GROUP LLP, 495 METRO PLACE SOUTH, SUITE 210, DUBLIN, OH, 43017, US

(73) Assignee: The Ohio State University Research Foundation, Columbus, OH

(21) Appl. No.: 10/436,879

(22) Filed: May 13, 2003

Related U.S. Application Data
(60) Provisional application No. 60/380,213, filed on May 13, 2002.

(51) Int. Cl. A61B 5/05

(52) U.S. Cl. 600/426

(57) ABSTRACT

An instrument-holding projection imaging vector guide and method of use is provided herein. In one embodiment of the present invention, a instrument-holding projection imaging vector guide is provided comprising a radio-opaque skin entry point member, a frame member and a diaphragm. The radio-opaque skin entry member is used to determine a skin entry point. The diaphragm is used to determine a diaphragm entry point. The radio-opaque skin entry point member and the diaphragm together determine the vector along which an appropriate medical instrument is to follow in order to contact a target inside a patient's body.

The present application claims priority to U.S. provisional application No. 60/380,213, filed May 13, 2002, which is hereby incorporated in its entirety by reference.

### TECHNICAL FIELD OF THE INVENTION

This invention relates generally to apparatus and methods useful in scientific research and interventional medicine, and useful in the visualization and analysis of organic tissues and bodies; and to research into the cause and symptoms of disease, its diagnosis and treatment. The invention particularly concerns apparatus that may be advantageously utilized by a researcher, physician or health care professional, in cooperation with fluoroscopy, mammography, and plain radiography.

### BACKGROUND OF THE INVENTION

Radiology is the discipline of medical science that uses electromagnetic radiation and ultrasonics for the diagnosis and treatment of injury and disease. Radiology originated with the discovery of X-rays by German physicist Wilhelm Conrad Roentgen in 1895. Roentgen was awarded the first Nobel Prize in physics (1901) for his work.

Diagnostic radiology, or diagnostic imaging, is the medical evaluation of body tissues and functions—both normal anatomy and physiology and abnormalities caused by disease or injury—by means of static or dynamic radiologic images.

In the century since Roentgen's discovery, electromagnetic radiation in the form of ionizing radiation (alpha, beta, gamma and X-rays) has been the predominant energy source for diagnostic radiology.

The use of ionizing radiation in diagnostic radiology involves passing a localized beam of X-rays through the part of the body being examined. This produces a static image on film. The image, called a radiograph, or X-ray picture, can take several forms. It may be a plain radiograph, such as the common chest X-ray; a mammogram, an X-ray image of the female breast used to scan for cancerous tumors; a tomograph, which produces an image of the entire depth of an anatomical structure with a series of X-rays; or a computerized axial tomography (CT or CAT) scan, a computer analysis of a cross-sectional image of the body. The image produced by any of these methods may be stored on film or in electronic format (such as a graphic file on a personal computer).

Many organs, organ systems, and certain muscular and skeletal structures are not visible with routine radiographic techniques. They become visible with the ingestion, injection, or inhalation of substances called contrast media, which are opaque to radiation. Diagnostic techniques involving contrast media include the upper gastrointestinal (GI) series, barium enemas (colon examination), arthrogram (injection of contrast into a joint), myelogram (injection of contrast into the spinal canal), and angiogram (injection of contrast into an artery, vein, or lymph vessel).

Dynamic images, which record movement of organs or organ systems such as the intestinal tract or the flow of contrast material through blood vessels or the spinal canal, may be obtained by fluoroscopy (recording the radiographic image on a movable, radiation-sensitive screen) or cineradiography (recording the image on film or videotape). Both film and videotape are permanent recording media. The fluoroscopic image, analogous to a television image, is transient. Permanent radiographic images (spot films) can, however, be made at any time during a fluoroscopic examination.

The use of ionizing radiation in the evaluation of disease is similar to the use of drugs in the treatment of disease. Diagnostic radiographic examinations should only be performed for a specific medical indication on the direct request of a physician or other qualified person. Although diagnostic radiation dosage levels involve a small health risk, there is no evidence to show detectable adverse effects of radiation from medically indicated and properly conducted diagnostic radiographic examinations. In the opinion of the American College of Radiology and the bureau of Radiologic Health of the United States Department of Health and Human Services, with careful patient selection, the risk-benefit ratio clearly lies in favor of the radiological procedure.

In a projection imaging procedure, such as fluoroscopy or mammography, the patient is placed under an imaging source (such as radiation for fluoroscopy, mammography, and plain radiographs, or sound waves for an ultrasound) to generate a real-time image of an area of the patient on a video monitor. The doctor may then use the image projected on the video monitor to align a radio-opaque medical instrument (such as a biopsy needle) with a target (such as a tumor) under the surface of the patient's skin. Traditionally, after the doctor had determined the skin entry position for the medical instrument, the doctor would insert the medical instrument without the use of mechanical assistance into the patient along a predetermined theoretical vector. However, the doctor had to rely on his manual dexterity to ensure that the actual vector followed by the medical instrument was the same as the theoretical one. Regrettably, this may be very difficult to do. Thus, there has been a long felt need for a device that can be used with projection imaging modalities such as fluoroscopy, mammography, plain radiography and ultrasound, that would assist a doctor in not only determining both an appropriate skin entry point and an appropriate vector for a medical instrument, but also in maintaining that vector as the medical instrument is advanced to the target. Obviously, the medical field demands that such a device be cost-effective, easy to use and assist in minimizing the amount of radiation exposure to medical staff and patient alike.

### SUMMARY OF THE INVENTION

The present invention provides a cost-effective, easy to use, projection imaging vectoring guide that can quickly assist a doctor, or other medical person, in determining an appropriate skin entry point and an appropriate vector for a medical instrument to follow. The present invention additionally supports the medical instrument in the chosen vector alignment position. Also provided is a method for using a projection imaging vectoring guide of the present invention.

The present invention provides a projection imaging vectoring guide comprising a radio-opaque skin entry point member; a frame member; and a diaphragm. The radio-opaque skin entry point member is adapted to be placed directly on the skin (surface) of a patient so as to define a skin entry point. The frame member has a first end and a second end. The first end of the frame member is adapted to be placed on the skin of the patient. The second end of the frame member is adapted to carry a diaphragm. As used herein, the term “carry” shall be understood as meaning either fixedly or removably having mounted thereto. The diaphragm is adapted to be carried by the frame member and may be secured to the frame member by mechanical means (such as clips or staples) or by an adhesive. Alternatively, the frame member and the diaphragm may be combined into a single piece without deviating from the spirit and scope of the present invention.

In a preferred embodiment of the present invention, the radio-opaque skin entry point member prohibits the passage of radiation thus appearing as a black form on the imaging monitor. It is preferred that the radio-opaque skin point entry member comprise a substrate upon which is disposed a radio-opaque substance. In a preferred radio-opaque skin entry point member, the radio-opaque substance is formed into a shape so as to define a skin entry point. Suitable shapes include cross-hairs, rings, hollow (unfilled) boxes, hollow triangles, or any other suitable shape. The substrate is preferably adapted to be affixed directly upon the skin of the patient either by having an adhesive disposed thereon or through other means such as the use of adhesive strips, clips, stitches, or staples.

In a preferred embodiment of the present invention, the frame member is constructed so as to provide access to the skin entry point with a medical instrument, such as a needle. Preferred forms for the frame member include hollow tubes, hollow cubes, or any other suitable form. It is preferred that the frame member is constructed from a lightweight, low cost material such as plastic. In a more preferred embodiment of the present invention, the frame member comprises two components: a lower frame portion and an upper frame portion. In such an embodiment, the lower frame portion and upper frame portion are adapted to removably mate with one another so as to permit the user to affix the lower frame portion to a patient prior to adding the upper frame portion so as to allow easy access to the skin entry point during the procedure. The lower frame portion is preferably further adapted to be fixedly placed on the patient. The lower frame portion may be attached to the patient by adhesive tape, clips or any other suitable means. The upper frame portion is adapted to carry a diaphragm. Further, it is preferred that the frame member (or the lower frame portion, depending upon the style of frame member employed) comprises a drape material.

The frame member carries the diaphragm. The diaphragm is preferably constructed from a lightweight, low cost material that is perforable by a wide range of diagnostic and/or therapeutic medical instruments such as needles, drainage tubes, fiber optics, lasers, and probes. It is preferred that the diaphragm be constructed of a material that when pierced, retains the shape of the piercing such that minimal force is needed to overcome sliding friction arising between the diaphragm and a medical instrument passing therethrough. Further, it is preferred that the diaphragm be constructed of a material that is sufficiently dimensionally stable to hold a wide variety of medical instruments in place without stretching, twisting or otherwise deforming the diaphragm due to the weight of the instrument. While the first point for the vector is determined by the radio-opaque skin entry point member, the diaphragm provides the second point for the vector, thereby defining the vector along which a medical instrument should be advanced to encounter the target.

Optionally, the diaphragm may be attached to the frame member by a hinge. The hinge may allow the diaphragm to be moved so as to provide access to the patient's skin in the region defined by the frame member. Thus, the hinge provides a means by which a doctor, or other medical professional, may gain access to the patient's skin (for example to sterilize the skin or to wipe blood from the skin) without having to remove the frame member.

A second option is for the frame member to have at least one access window. An access window is an opening in the frame member through which access to the patient's skin may be gained. Similar to the hinge, an access window provides a means by which a doctor, or other medical professional, may gain access to the patient's skin (for example to sterilize the skin or to wipe blood from the skin) without having to remove the frame member.

The present invention also provides an instrument holding device comprising a frame member and a diaphragm. The frame member comprises a first end and a second end. The first end is adapted to be placed on a patient's skin. The second end of the frame member is adapted to carry a diaphragm. The diaphragm comprises a perforable membrane adapted to provide support to an instrument passing therethrough. In practice, an instrument holding device of the present invention assists the medical professional by providing a device that can support an instrument during a procedure such that the medical professional may let go of the instrument without fear that the instrument will move or fall out of the patient.

In preferred embodiments of the instrument holding device, the frame member additionally comprises at least one access window. Each access window permits at least partial access to the patient's skin under the frame member without having to remove the frame member. In yet another preferred embodiment, the instrument-holding device additionally comprises a hinge adapted to permit the diaphragm to be moved so as to provide at least partial access to the patient's skin under the frame member.

A preferred method for using a projection imaging vectoring guide of the present invention comprises the steps of: (1) affixing a radio-opaque skin entry point member on the patient's skin so as to define a skin entry point, the skin entry point defining one point along a vector; (2) affixing a frame member to the patient, the frame member having a first and second end, wherein the second end is adapted to carry a diaphragm, the frame member being positioned above the radio-opaque skin entry point member so as to position at least a portion of the diaphragm over the radio-opaque skin entry point member; (3) determining the location of a diaphragm entry point using a radio-opaque instrument, the diaphragm entry point defining a second point along the vector; (4) piercing the diaphragm so as to create a diaphragm entry point; and (5) advancing a medical instrument along the defined vector by going through the diaphragm entry point and skin entry point so as to contact a target inside the patient. Obviously, the vector may be adjusted by manually manipulating the frame member while out of the radiation beam. Further, the radio-opaque instrument used to determine the diaphragm entry point may be a radio-opaque instrument such as a needle or other appropriate device.

A preferred method of the present invention further comprises the additional step of sterilizing the patient so as to avoid infection resulting from the procedure. That is to say, the patient's skin is sterilized about the area where the skin entry point is located so as to reduce the chance of infection. In a more preferred method, the method further comprises the additional step of using projection imaging to determine the location of the skin entry point. Further yet, a more preferred method comprises the additional step of using projection imaging to determine the diaphragm entry point.

### BRIEF DESCRIPTION OF THE DRAWINGS

FIG. 1 illustrates one embodiment of a radio-opaque skin entry point member of the present invention.

FIG. 2 illustrates a frame member and diaphragm of one embodiment of the present invention.

FIG. 3 illustrates one embodiment of a lower frame portion of a frame member of the present invention.

FIG. 4 illustrates one embodiment of an upper frame portion of a frame member of the present invention with the diaphragm attached.

FIG. 5 shows the assembly of the lower frame portion and the upper frame portion in accordance with one embodiment of the present invention.

FIG. 6 illustrates a radio-opaque skin entry point member and lower frame portion placed on a patient.

FIG. 7 shows the use of a radio-opaque instrument to locate a diaphragm entry point.

FIG. 8 shows a probe being inserted through the diaphragm entry point and the skin entry point with forceps being used to stabilize the diaphragm and provide fine adjustment to the vector.

### DETAILED DESCRIPTION OF THE PREFERRED EMBODIMENT(S)

In accordance with the foregoing summary, the following presents a detailed description of the preferred embodiment of the invention that is currently considered to be the best mode.

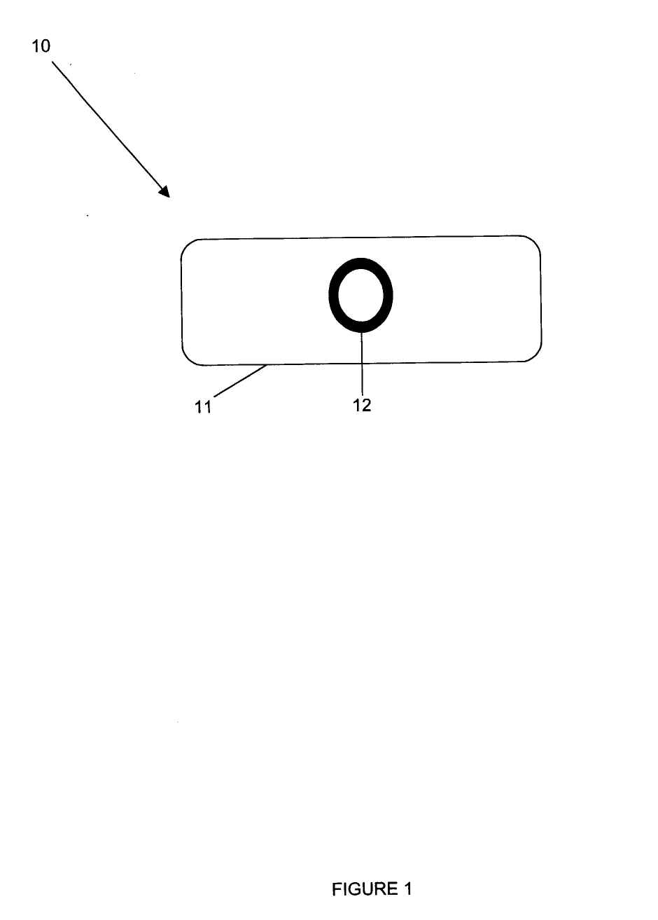

FIG. 1 shows one embodiment of a radio-opaque skin entrypoint member 10. The radio-opaque skin entry point member 10 comprises a substrate 11 and a radio-opaque substance 12. The substrate 11 may be constructed from a perforated or perforable material or may have material removed so as to provide an opening to the patient's skin. The radio-opaque substance 12 is deposited on the substrate 11 so as to define a skin entry point for a medical instrument. In instances where the substrate has an opening to the patient's skin, the radio-opaque material is disposed about the opening so as to define its edges. The substrate 11 may be provided with an adhesive backing so as to facilitate attachment to a patient's skin. In this regard, the radio-opaque skin entry point member would adhere to the patient's skin in much the same manner as a BAND-AID.

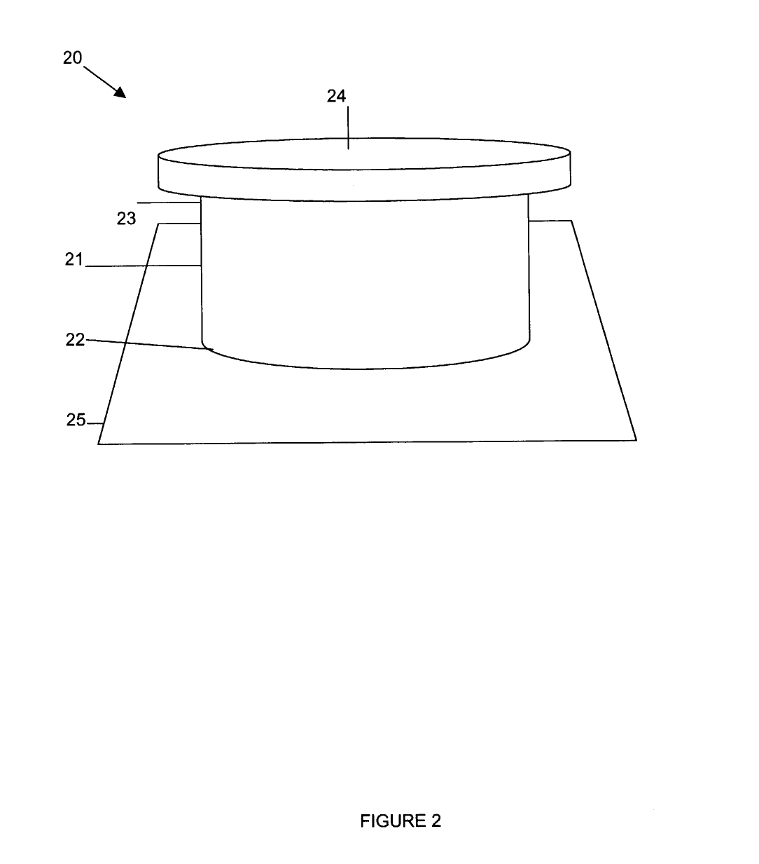

FIG. 2 illustrates the combination 20 of a frame member 21 and diaphragm 24 of one embodiment of the present invention. The frame member 21 comprises a first end 22 and a second end 23. Also shown is the optional drape 25. The diaphragm 24 is carried by frame member 21 so as to define a platform above the surface of the patient where a second point along a vector may be defined.

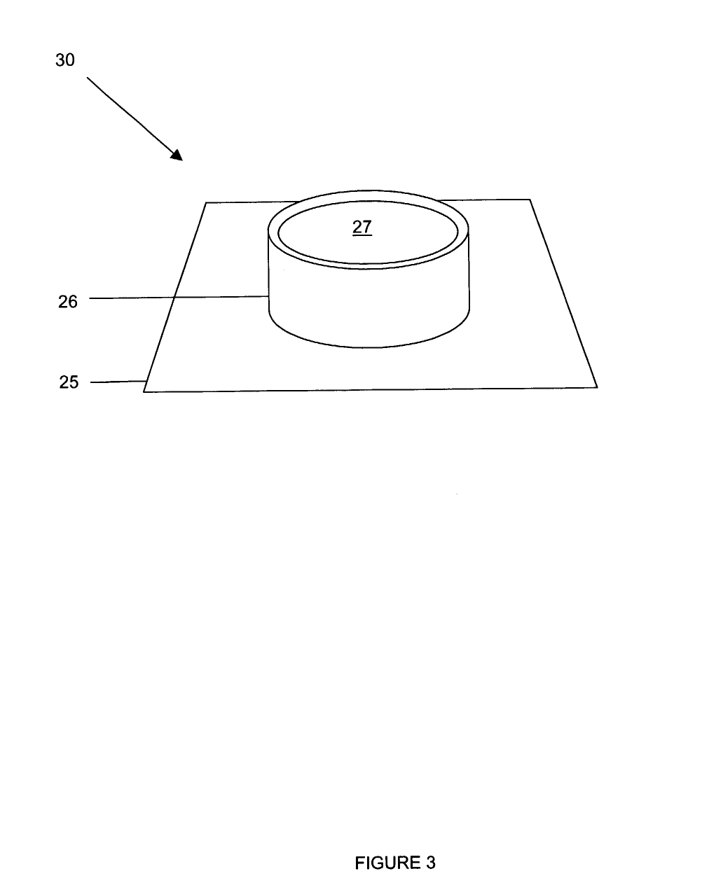

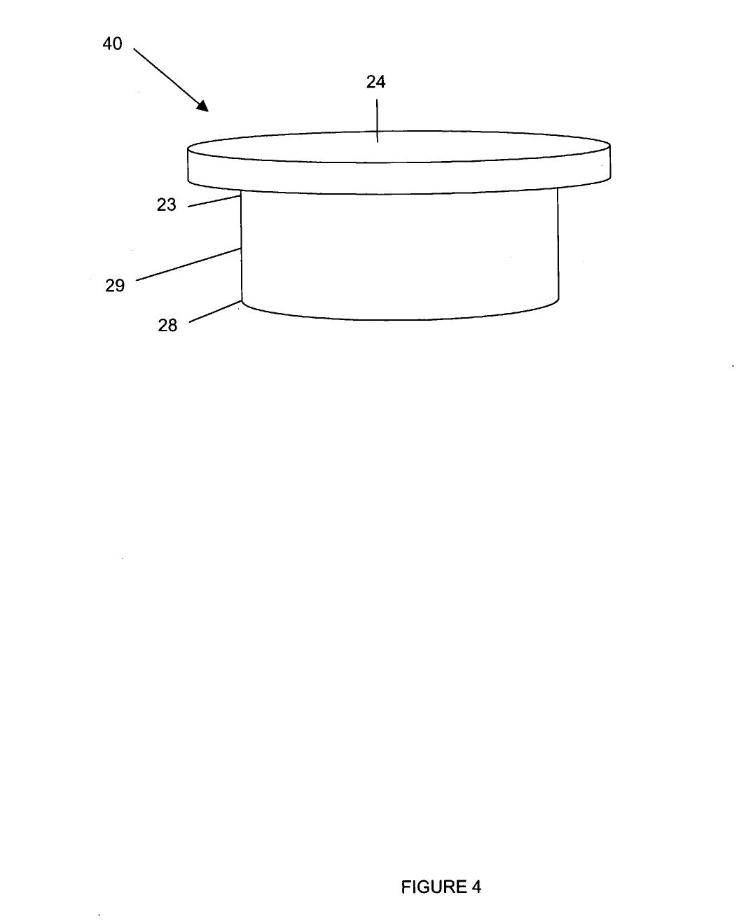

FIG. 3 illustrates the combination 30 of a lower frame portion 26 and drape 25 in accordance with one embodiment of the present invention. As discussed above, the frame member 21 may be constructed from two pieces, a lower frame portion 26 and an upper frame portion 29 (shown in FIG. 4), that are designed to removably mate with one another. The lower frame portion 26 is adapted to be placed on the surface of the patient. The skin entry point should be visible inside hollow region 27. The lower frame portion 26 mates with a corresponding upper frame portion 29, as shown in FIG. 4.

FIG. 4 shows the combination 40 of an upper frame portion 29 and diaphragm 24. Upper frame portion 29 is adapted to removably mate with lower frame portion 26 (shown in FIG. 3). Upper frame portion 29 has a first end 28 and a second end 23. The second end 23 of upper frame portion 29 carries diaphragm 24. Diaphragm 24 is constructed from an appropriate perforable material so as to allow passage there through of a medical instrument. Further, diaphragm 24 may be constructed from an appropriate perforable material so as to hold a medical instrument in position during a procedure.

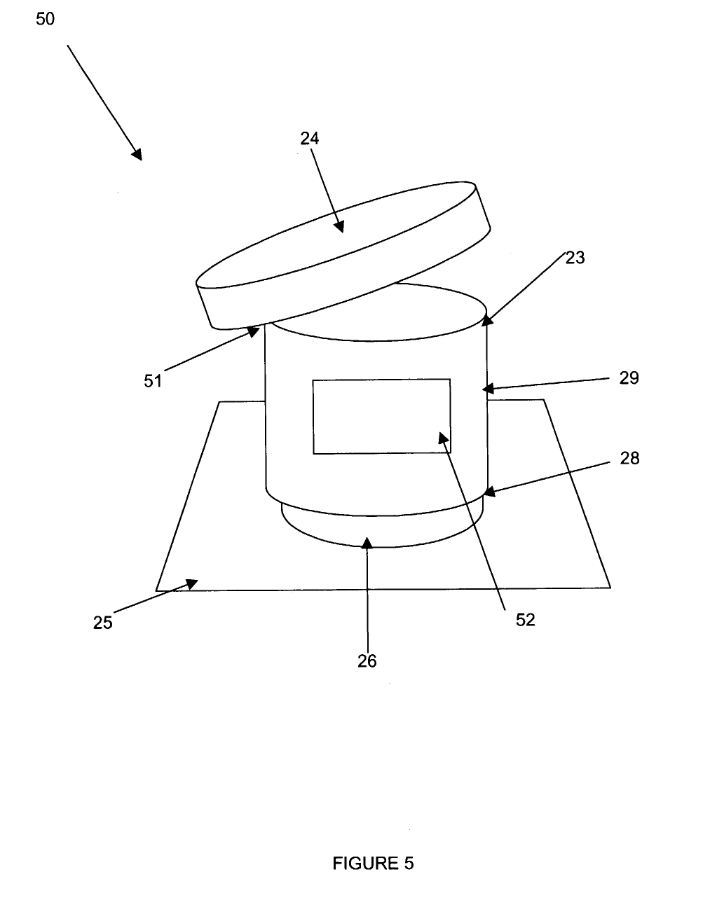

FIG. 5 shows the assembly 50 of combination 30 and combination 40 in accordance with one embodiment of the present invention. FIG. 5 further shows optional hinge 51 and access window 52. Hinge 51 may be constructed in a variety of styles and materials. In a top opening hinge, as shown in FIG. 5, it is preferred that hinge 51 permits diaphragm 24 to swing through an arc of at least 90 degrees. Alternatively, a diaphragm may be rotated about a vertically disposed hinge so as to displace the diaphragm horizontally. That is to say, the diaphragm “swings away” from the frame member, thereby granting access to hollow region 27.

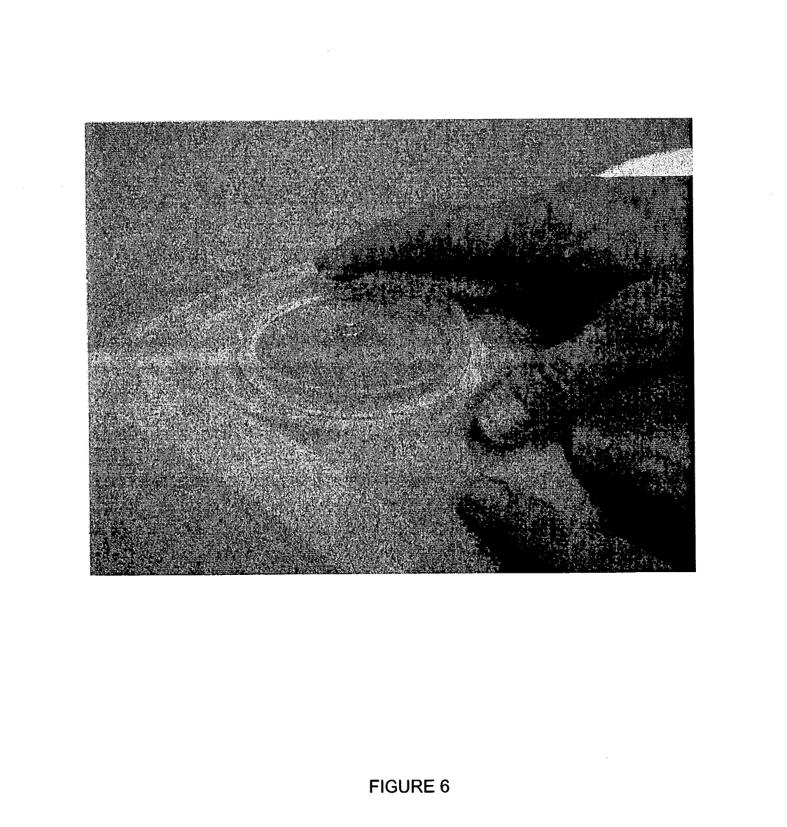

FIG. 6 shows a radio-opaque skin entry point member 10 and the combination 30 of a lower frame portion 26 and drape 25 placed on the surface of a patient. The skin entry point is determined using projection imaging. After placing the radio-opaque skin entry point member 10 appropriately over the skin entry point, the lower frame portion 26 is placed over the skin entry point. As shown, the skin entry point may be centered in the lower frame portion. Next, combination 40, comprising the upper frame portion 29 and the diaphragm 24, is seated on lower frame portion 26, thereby completing the instrument-holding projection imaging vector guide of the present invention.

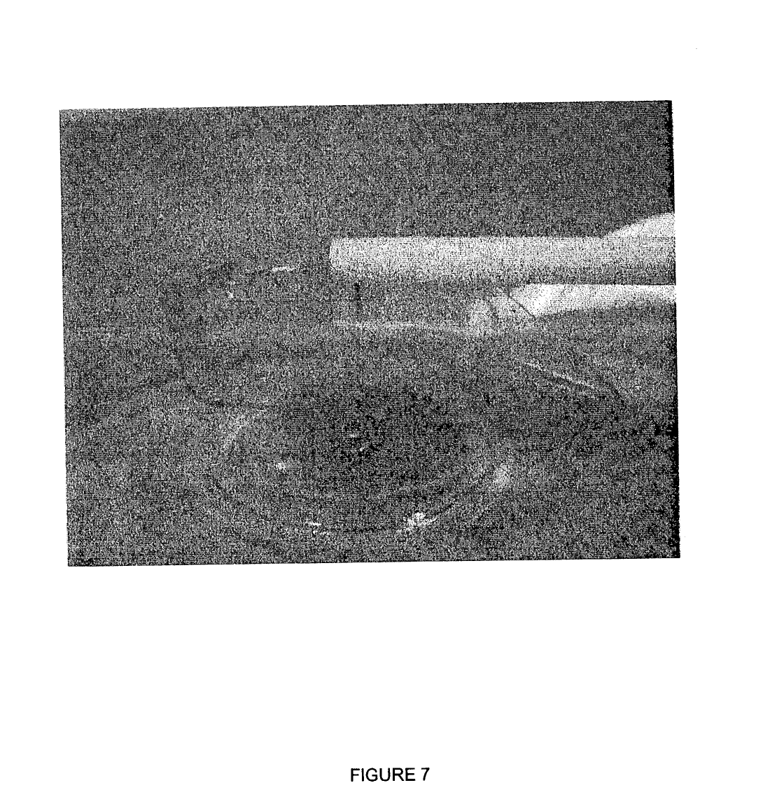

FIG. 7 shows the instrument-holding projection imaging vector guide on the surface of a patient. The instrument-holding projection imaging vector guide comprises the radio-opaque skin entry point member 10, frame member 21, and diaphragm 24. Also shown in FIG. 7 is a radio-opaque instrument that is used to identify a diaphragm entry point in conjunction with projection images. The radio-opaque skin entry point member may be a pen, a pin, a needle, a wire or any appropriate radio-opaque implement. Once the diaphragm entry point is determined, the diaphragm 24 is pierced thereby forming the diaphragm entry point.

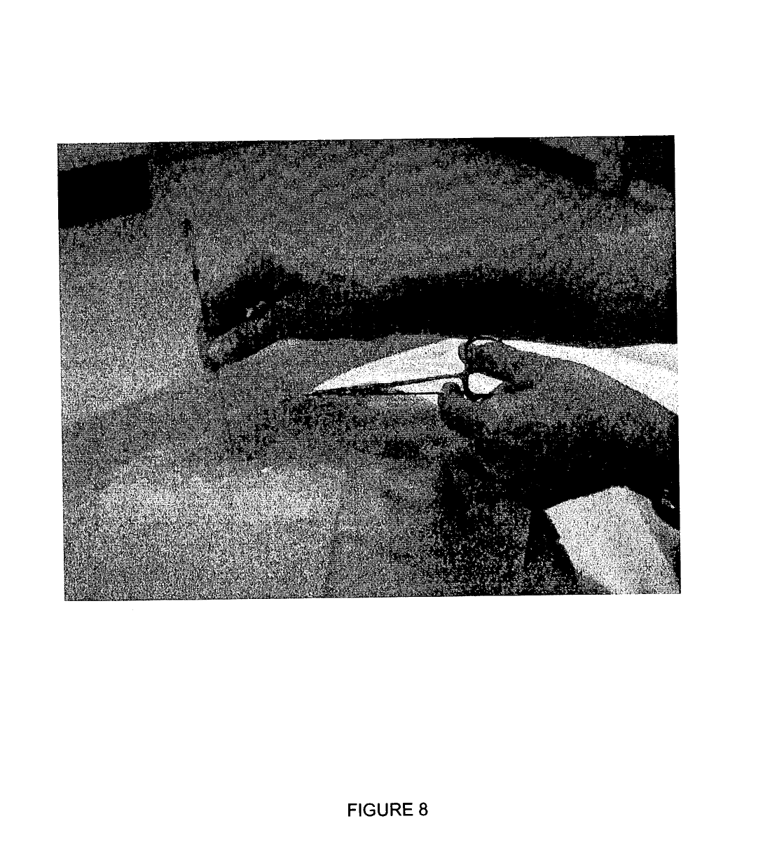

FIG. 8 shows the advancement of a medical instrument through the diaphragm entry point and the skin entry point. The forceps shown may be used to stabilize the instrument-holding projection imaging vector guide, thereby keeping it from moving. The forceps may also be used to make subtle adjustments in the vector defined by the position of the skin entry point and the position of the diaphragm entry point. An additional benefit of using forceps, or another appropriate implement, to manipulate the instrument-holding projection imaging vector guide is that the user is able to avoid direct contact with the radiation beam.

The preferred embodiments herein disclosed are not intended to be exhaustive or to unnecessarily limit the scope of the invention. The preferred embodiments were chosen and described in order to explain the principles of the present invention so that others skilled in the art may practice the invention. Having shown and described preferred embodiments of the present invention, it will be within the ability of one of ordinary skill in the art to make alterations or modifications to the present invention, such as through the substitution of equivalent materials or structural arrangements, or through the use of equivalent process steps, so as to be able to practice the present invention without departing from its spirit as reflected in the appended claim, the text and teaching of which are hereby incorporated by reference herein. It is the intention, therefore, to limit the invention only as indicated by the scope of the claims and equivalents thereof.

What is claimed is:

1. An instrument-holding projection imaging vector guide, said instrument-holding projection imaging vector guide comprising:
a radio-opaque skin entry point member, said radio opaque skin entry point member adapted to be placed directly on the skin of a patient so as to define a skin entry point;
a frame member, said frame member having a first end adapted to be placed on said patient, said frame member having a second end adapted to carry an diaphragm; and
an diaphragm, said diaphragm comprising a perforable membrane.

2. The instrument-holding projection imaging vector guide of claim 1 wherein said radio-opaque skin entry point member comprises a radio-opaque member and an adhesive portion adapted to affix said radio-opaque member to said patient, wherein said radio-opaque member defines said skin entry point.

3. The instrument-holding projection imaging vector guide of claim 1 wherein said radio-opaque skin entry point member comprises an adhesive member having a radio-opaque pattern disposed thereon, said radio-opaque pattern defining said skin entry point

4. The instrument-holding projection imaging vector guide of claim 1 wherein said frame member comprises a lower frame portion and an upper frame portion, said lower frame portion and said upper frame portion adapted to removably mate with one another.

5. The instrument-holding projection imaging vector guide of claim 4 wherein said lower frame portion comprises a drape material.

6. The instrument-holding projection imaging vector guide of claim 4 wherein said lower frame portion is adapted to be fixedly placed on said patient.

7. The instrument-holding projection imaging vector guide of claim 4 wherein said upper frame portion is adapted to carry said diaphragm.

8. The instrument-holding projection imaging vector guide of claim 1 wherein said frame member is constructed of plastic.

9. The instrument-holding projection imaging vector guide of claim 1 wherein said diaphragm comprises a perforable plastic membrane.

10. The instrument-holding projection imaging vector guide of claim 1 additionally comprising a hinge, said hinge adapted to allow said diaphragm to be moved so as to provide access to said radio-opaque skin entry point member.

11. The instrument-holding projection imaging vector of claim 1 wherein said frame member additionally comprises at least one access window, each said access window permitting access to said skin entry point without having to remove said frame member.

12. An instrument holding device, said instrument holding device comprising:
a frame member, said frame member comprising a first end and a second end, said first end adapted to be placed on a patient, said second end adapted to carry a diaphragm;
a diaphragm, said diaphragm comprising a perforable membrane, said perforable membrane adapted so as to provide support to an instrument passing therethrough.

13. The instrument-holding device of claim 12, wherein said frame member additionally comprises at least one access window, each said access window permitting at least partial access to said patient's skin under said frame member without having to remove said frame member.

13. The instrument-holding device of claim 12 additionally comprising a hinge, said hinge adapted to allow said diaphragm to be moved so as to provide at least partial access to said patient's skin under said frame member.

14. A method for using an instrument-holding projection imaging vector guide on a patient, said method comprising the steps of:
affixing a radio opaque skin entry point member on said patient so as to define a skin entry point, said skin entry point defining one point along a vector;
affixing a frame member to said patient, said frame member having a first and second end, wherein said second end is adapted to carry a diaphragm, said frame member being positioned about said radio opaque skin entry point member so as to position at least a portion of said diaphragm over said radio opaque skin entry point member;
determining the location of an diaphragm entry point using a radio opaque device, said diaphragm entry point defining a second point along said vector;
piercing said diaphragm so as to create an diaphragm entry point; and
advancing an instrument along said vector by going through said diaphragm entry point and said skin entry point so as to contact a target inside said patient.

15. The method according to claim 14 additionally comprising the step of sterilizing said patient so as to avoid infection resulting from said method.

16. The method according to claim 14 additionally comprising the step of using projection imaging to determine said skin entry point.

17. The method according to claim 14 additionally comprising the step of using projection imaging to determine said diaphragm entry point.

18. The method according to claim 14 wherein said frame member comprises a lower frame portion and an upper frame portion, said lower frame portion adapted to removably mate with said upper frame portion.
